(19) United States
(12) Patent Application Publication
(10) Pub. No.: US 2026/0343074 A1
(43) Pub. Date: Oct. 29, 2026

(54) INFORMATION ACQUISITION METHOD, STORAGE MEDIUM, IMAGE ACQUISITION APPARATUS, AND INFORMATION ACQUISITION APPARATUS

(71) Applicant: CANON KABUSHIKI KAISHA, Tokyo (JP)

(72) Inventor: HIDEKI KATO, Tokyo (JP)

(21) Appl. No.: 19/704,544

(22) Filed: Jun. 11, 2026

Related U.S. Application Data
(63) Continuation of application No. PCT/JP2024/043984, filed on Dec. 12, 2024.

(30) Foreign Application Priority Data
Dec. 18, 2023 (JP) 2023-213063
Oct. 28, 2024 (JP) 2024-188835

(51) Int. Cl. H04N 23/11; G06T 5/50; G06T 5/70; G06V 10/24; G06V 10/60; G16H 30/20; G16H 30/40

(52) U.S. Cl. CPC H04N 23/11; G06T 5/50; G06T 5/70; G06V 10/24; G06V 10/60; G16H 30/20; G16H 30/40; G06T 2207/10064; G06T 2207/20182; G06T 2207/20221

(57) ABSTRACT

By a method including a fluorescence image acquisition step, an azimuth correction amount acquisition step, an azimuth-corrected image acquisition step, a template mask information acquisition step, a first translational correction amount acquisition step, a second translational correction amount acquisition step, and a fluorescence intensity acquisition step, information regarding fluorescence intensity with ensured reproducibility is acquired in a fluorescence image having morphological information of spots arranged on an array plate and a fluorescence image having functional information.

### CROSS-REFERENCE TO RELATED APPLICATIONS

This application is a Continuation of International Patent Application No. PCT/JP 2024/043984, filed Dec. 12, 2024, which claims the benefit of Japanese Patent Application No. 2023-213063, filed Dec. 18, 2023, and Japanese Patent Application No. 2024-188835, filed Oct. 28, 2024, all of which are hereby incorporated by reference herein in their entirety.

### BACKGROUND

### Field of the Technology

The present disclosure relates to an information acquisition method, a storage medium, an image acquisition apparatus, and an information acquisition apparatus.

### Description of the Related Art

Array plates are known, such as protein arrays, peptide arrays, and DNA arrays, in which a large number of substances such as proteins, peptides, and nucleic acids are fixed in spots on a substrate such as glass or plastic. By using an array plate, interactions between a large number of fixed substances and substances in a sample can be observed at once. Therefore, interactions between a large number of substances and various liquid samples including biologically derived specimens such as blood, cell extracts, saliva, and interstitial fluid are comprehensively analyzed.

In a measurement process of an array plate, a method is known in which spots where an interaction of interest has occurred are selectively fluorescently labeled to obtain optical information. Known measurement apparatuses for obtaining optical information include microarray scanners and plate readers. Some of these measurement apparatuses are known to have a function for exciting fluorescent dyes at a plurality of wavelengths, and in such measurement apparatuses, fluorescence images of an array plate at the respective excitation wavelengths are acquired. On the other hand, in an analysis process of an array plate, a method is known in which fluorescence information of each spot is acquired by using a template mask in which an analysis area is defined for a fluorescence image of the array plate. In Japanese Patent Laid-Open No. 2011-182705, the positional deviation between the template mask and the spots in the fluorescence image is automatically adjusted, and information regarding fluorescence intensity at each spot is acquired.

However, in Japanese Patent Laid-Open No. 2011-182705, when the fluorescence brightnesses of a large number of spots on an array plate are not sufficient, it is difficult to adjust the position of the template mask, and therefore it is also difficult to analyze fluorescence information of each spot.

That is, a measurement apparatus such as a plate reader capable of acquiring two-wavelength fluorescence images can be used to acquire a first fluorescence image imaged by irradiating first primary light having one wavelength, and this can be used as an image for confirming the shapes of spots and for adjusting the position of a template mask, and to acquire a second fluorescence image imaged by irradiating second primary light having a wavelength different from that of the first primary light, and this can be used as an image for acquiring functional information. However, the fluorescence brightnesses of a large number of spots on the array plate in the second fluorescence image may not be sufficient, and in such a case it is difficult to adjust the position of the template mask. Further, when there is a positional deviation between the first fluorescence image and the second fluorescence image, it is difficult to accurately analyze fluorescence information of each spot with a template mask aligned with the first fluorescence image.

### SUMMARY

The present disclosure is to provide an information acquisition method and an information acquisition apparatus that acquire information regarding fluorescence intensity with ensured reproducibility in a fluorescence image having morphological information of spots on an array plate and a fluorescence image having functional information.

The present disclosure provides an information acquisition method for acquiring information regarding fluorescence intensity of spots arranged on an array plate, including: a fluorescence image acquisition step of acquiring, with respect to an array plate provided with a plurality of arranged spots, a first fluorescence image imaged by irradiating first primary light, and a second fluorescence image imaged by irradiating second primary light having a wavelength different from that of the first primary light in order to acquire functional information of the spots; an azimuth correction amount acquisition step of acquiring information regarding an azimuth correction amount within the image plane of the first fluorescence image and the second fluorescence image based on the first fluorescence image; an azimuth-corrected image acquisition step of acquiring a first azimuth-corrected image obtained by correcting an azimuth with respect to the first fluorescence image and a second azimuth-corrected image obtained by correcting an azimuth with respect to the second fluorescence image based on the information regarding the azimuth correction amount; a template mask information acquisition step of acquiring information regarding a template mask including a plurality of first regions arranged corresponding respectively to the plurality of spots, wherein a reference position and relative positions defining an outer edge with respect to the reference position are defined so that one of the plurality of spots overlaps a corresponding first region and another adjacent spot does not overlap the first region; a first translational correction amount acquisition step of acquiring information regarding a first translational correction amount for the plurality of first regions included in the first azimuth-corrected image by adjusting a relative position of the template mask with respect to the first azimuth-corrected image; a second translational correction amount acquisition step of acquiring a second translational correction amount for the plurality of first regions included in the second azimuth-corrected image by adjusting a relative position of the template mask with respect to the second azimuth-corrected image based on the information regarding the first translational correction amount for the plurality of first regions included in the first azimuth-corrected image; and a fluorescence intensity acquisition step of acquiring information regarding fluorescence intensity corresponding to the plurality of spots in the first fluorescence image based on the information regarding the first translational correction amount and the first azimuth-corrected image, and acquiring information regarding fluorescence intensity corresponding to the plurality of spots in the second fluorescence image based on the information regarding the second translational correction amount and the second azimuth-corrected image, thereby solving the above problem.

Features of the present disclosure will become apparent from the following description of embodiments with reference to the attached drawings.

### BRIEF DESCRIPTION OF THE DRAWINGS

FIG. 1 is a diagram illustrating an information acquisition method S10000 according to the first embodiment.

FIG. 2 is a schematic diagram of an array plate applied to the information acquisition method according to the first embodiment.

FIG. 3A is a diagram showing an example of a first fluorescence image obtained in the fluorescence image acquisition step S100 in the first embodiment.

FIG. 3B is a diagram showing an example of a second fluorescence image obtained in the fluorescence image acquisition step S100 in the first embodiment.

FIG. 4A is a diagram illustrating the azimuth correction amount acquisition step S200 in the first embodiment.

FIG. 4B is a diagram illustrating the azimuth correction amount acquisition step S200 in the first embodiment.

FIG. 5 is a schematic diagram of a template mask obtained in the template mask information acquisition step S400 in the first embodiment.

FIG. 6A is a diagram illustrating the first translational correction amount acquisition step S500 in the first embodiment.

FIG. 6B is a diagram illustrating the first translational correction amount acquisition step S500 in the first embodiment.

FIG. 7A is a diagram illustrating the second translational correction amount acquisition step S600 in the first embodiment.

FIG. 7B is a diagram illustrating the second translational correction amount acquisition step S600 in the first embodiment.

FIG. 8 is a schematic diagram of a second region in the fluorescence intensity acquisition step S700 in the first embodiment.

FIG. 9A is a diagram illustrating the first translational correction amount acquisition step S500 in the third embodiment.

FIG. 9B is a diagram illustrating the first translational correction amount acquisition step S500 in the third embodiment.

FIG. 10A is a diagram illustrating the second translational correction amount acquisition step S600 in the third embodiment.

FIG. 10B is a diagram illustrating the second translational correction amount acquisition step S600 in the third embodiment.

FIG. 11 is a diagram illustrating the noise processing step SS220 in the fifth embodiment.

FIG. 12 is a diagram illustrating the spot extraction processing step SS240 (S203) in the fifth embodiment.

FIG. 13A is a diagram showing an example of an image acquired in the region removal processing step S206 in the fifth embodiment.

FIG. 13B is a diagram showing an example of an image acquired in the region removal processing step S206 in the fifth embodiment.

FIG. 14 is a diagram illustrating the composite image acquisition step SS260 in the sixth embodiment.

FIG. 15 is a diagram showing an example of a schematic configuration of an image acquisition apparatus 101 for implementing the information acquisition method according to the first embodiment.

FIG. 16 is a diagram showing an example of a schematic configuration of the image acquisition apparatus 101 for implementing the information acquisition method according to the first embodiment in a different form.

FIG. 17 is a diagram showing a schematic configuration of an information acquisition apparatus 11000 for implementing the information acquisition method according to the first embodiment.

### DESCRIPTION OF THE EMBODIMENTS

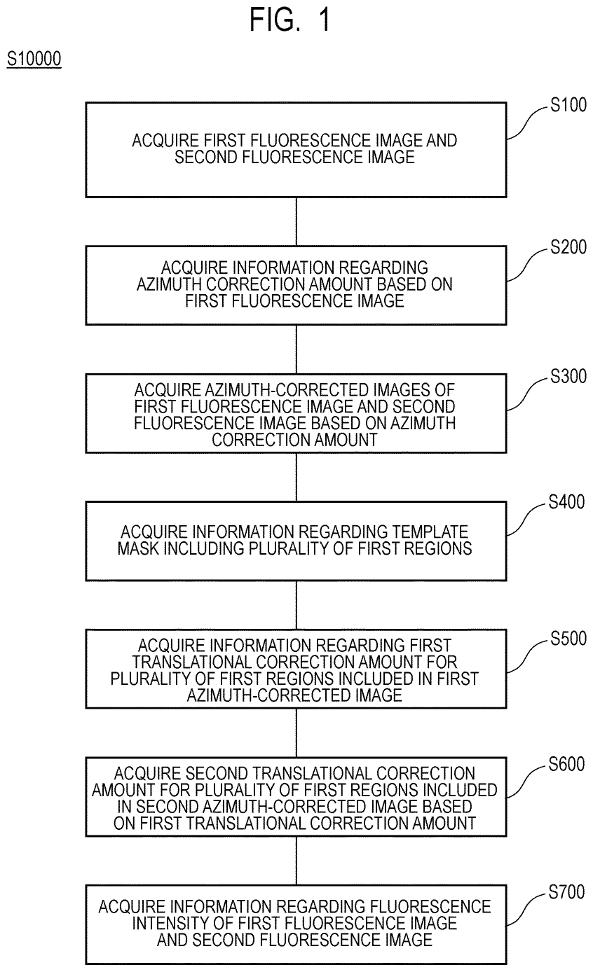

Hereinafter, an information acquisition method of the first embodiment of the present disclosure will be described using process diagram S10000 shown in FIG. 1. The information acquisition method of the first embodiment of the present disclosure is an information acquisition method for a fluorescence image of an array plate provided with a plurality of arranged spots, and includes the following steps.

A first fluorescence image imaged by irradiating first primary light and a second fluorescence image imaged by irradiating second primary light having a wavelength different from that of the first primary light in order to acquire functional information of the spots are acquired (fluorescence image acquisition step S100).

Information regarding an azimuth correction amount within the image plane of the first fluorescence image and the second fluorescence image is acquired based on the first fluorescence image (azimuth correction amount acquisition step S200).

Based on the information regarding the azimuth correction amount, a first azimuth-corrected image obtained by correcting the azimuth of the first fluorescence image and a second azimuth-corrected image obtained by correcting the azimuth of the second fluorescence image are acquired (azimuth-corrected image acquisition step S300).

Information regarding a template mask including a plurality of first regions arranged corresponding respectively to the plurality of spots is acquired, wherein a reference position and relative positions defining an outer edge with respect to the reference position are defined so that one of the plurality of spots overlaps a corresponding first region and another adjacent spot does not overlap the first region (template mask information acquisition step S400).

By adjusting the relative position of the template mask with respect to the first azimuth-corrected image, information regarding a first translational correction amount for a plurality of first regions included in the first azimuth-corrected image is acquired (first translational correction amount acquisition step S500).

By adjusting the relative position of the template mask with respect to the second azimuth-corrected image based on information regarding the first translational correction amount, a second translational correction amount for a plurality of first regions included in the second azimuth-corrected image is acquired (second translational correction amount acquisition step S600).

Information regarding fluorescence intensity corresponding to a plurality of spots in the first fluorescence image is acquired based on the information regarding the first translational correction amount and the first azimuth-corrected image, and information regarding fluorescence intensity corresponding to a plurality of spots in the second fluorescence image is acquired based on the information regarding the second translational correction amount and the second azimuth-corrected image (fluorescence intensity acquisition step S700).

Details of each step will be described later.

### Array Plate

An array plate has spots containing a wide variety of biomaterials on a substrate, and is used for comprehensive analysis of specimens. An array plate is also sometimes called a microchip, a microarray, a protein chip, a DNA chip, or the like.

A commercially available array plate may be used as the array plate. Microarray plates are sold by Agilent Technologies Inc., RayBiotech, and the like. Alternatively, an array plate is produced with reference to a known method. That is, an array plate is produced by immobilizing a desired biomaterial on one surface of an appropriate substrate. Note that immobilization as used here may also be referred to as adsorption, and includes immobilization by hydrophobic interaction, electrostatic interaction, van der Waals interaction, hydrogen bonding, and covalent bonding. The substrate is preferably transparent. Examples of materials of the substrate include glass, synthetic quartz, quartz, borosilicate glass, and the like. Alternatively, examples of materials of the substrate include resins such as polystyrene, polypropylene, (meth)acrylic resin, polyamide, polyimide, melamine, ABS, polyphenylene oxide urethane, silicone, epoxy, and polydimethylsiloxane.

### First Fluorescence Image

The first fluorescence image is an image imaged by irradiating first primary light. The first fluorescence image is morphological information of spots on an array plate.

For example, when a measurement target is a protein and a plurality of kinds of proteins are spotted in an array on an array plate, GST-TAGs are attached to all of the spotted proteins, and the GST-TAGs are labeled with a fluorescent body that emits fluorescence in response to light of the first wavelength. When this is excited with light of the first wavelength, fluorescence from all of the proteins can be acquired, and as a result morphological information can be acquired.

In general, protein spots are transparent to visible light and are difficult to observe with an ordinary optical microscope, and therefore fluorescent labeling is an effective method.

### Second Fluorescence Image

The second fluorescence image is an image having functional information of spots on an array plate, the image being imaged by irradiating second primary light having a wavelength different from that of the first primary light.

By reacting an array plate with a biologically derived specimen, a part of the spotted proteins is phosphorylated, and the phosphorylated proteins are labeled with a fluorescent body that emits fluorescence in response to light of the second wavelength. When this is excited with light of the second wavelength, fluorescence from the phosphorylated proteins can be acquired, and as a result functional information (phosphorylation information) can be acquired.

In the present specification, light of the first wavelength may also be referred to as first primary light of the first wavelength, excitation light of the first wavelength, or first primary light. Similarly, light of the second wavelength may also be referred to as second primary light of the second wavelength, excitation light of the second wavelength, or second primary light.

In the present specification, light of the first wavelength may also be referred to as first primary light of the first wavelength, excitation light of the first wavelength, or first primary light. Similarly, light of the second wavelength may also be referred to as second primary light of the second wavelength, excitation light of the second wavelength, or second primary light.

To implement the information acquisition method of the present embodiment, an image acquisition apparatus is used, the image acquisition apparatus having an optical system including an irradiation unit that irradiates the array plate with primary light and a light-collection unit that collects secondary light from the spots, a placement unit on which the array plate is placed, a scanning unit that relatively moves the placement unit and the optical system, a control unit that controls scanning by the scanning unit and irradiation timing of the optical system, and a computer that executes a program causing each step of the information acquisition method of the present embodiment to be executed.

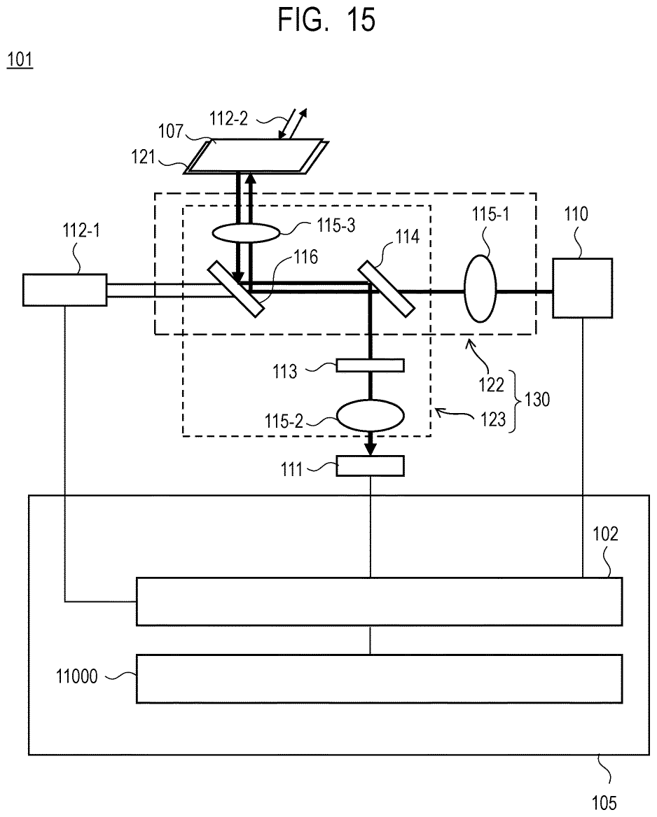

The above image acquisition apparatus will be described with reference to FIGS. 15 to 17.

FIG. 15 is a diagram showing a schematic configuration of an image acquisition apparatus 101 for implementing the information acquisition method according to the present embodiment. The image acquisition apparatus 101 includes an apparatus control unit 102, a placement unit 121 on which an array plate 107 is placed, a scanning unit 112-1 that scans the optical system, a scanning unit 112-2 that scans the placement unit 121, an irradiation unit 122 that irradiates the array plate 107 with primary light, a light-collection unit 123 that collects secondary light from the array plate 107, and a computer 105.

The irradiation unit 122 includes a lens 115-1, a dichroic mirror 114, a half mirror 116, and an objective lens 115-3. The irradiation unit 122 is optically coupled to the light source 110. Note that the light source 110 is constituted by an excitation light source. The image acquisition apparatus 101 may include the light source 110.

The light-collection unit 123 includes a filter 113, a lens 115-2, a dichroic mirror 114, a half mirror 116, and an objective lens 115-3.

The optical system 130 includes the irradiation unit 122 and the light-collection unit 123. The irradiation unit 122 and the light-collection unit 123 share a part of the optical system 130 including the dichroic mirror 114, the half mirror 116, and the objective lens 115-3, as elements in a region where the two dashed rectangles overlap in FIG. 15. That is, the optical system 130 includes the filter 113, the lens 115-1, the lens 115-2, the objective lens 115-3, the dichroic mirror 114, and the half mirror 116.

The irradiation unit 122 is optically coupled to the light source 110 via the lens 115-1 provided in the irradiation unit 122. The light-collection unit 123 is optically coupled to the photodetector 111 via the lens 115-2 provided in the light-collection unit 123.

The computer 105 has an information acquisition apparatus 11000 that executes a program for executing steps S100 to S700 of the information acquisition method S10000 of the present embodiment, and an apparatus control unit 102 that controls the placement unit 121, the irradiation unit 122, and the light-collection unit 123 that constitute the optical imaging system. That is, the information acquisition apparatus 11000 is an element constituting the image acquisition apparatus 101. The array plate 107 may also be referred to as a target that provides an image to be acquired by the image acquisition apparatus 101.

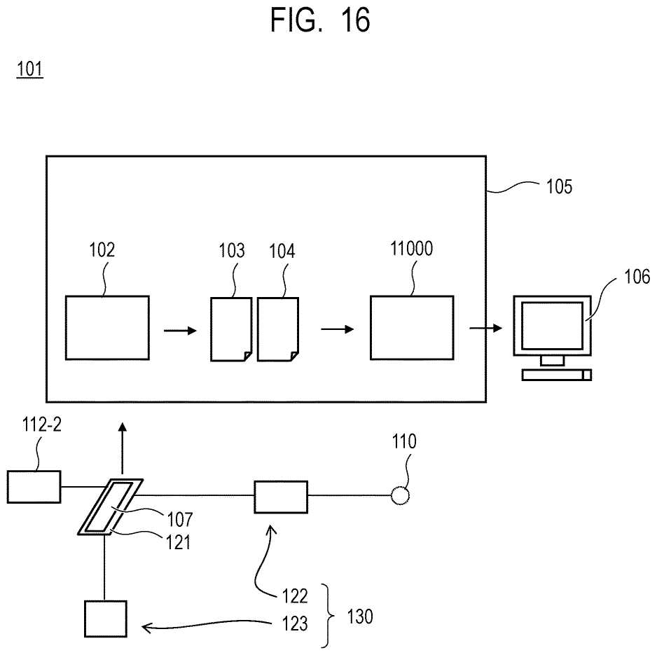

FIG. 16 is a diagram showing a schematic configuration of the image acquisition apparatus 101 for implementing the information acquisition method according to the present embodiment in a form different from FIG. 15.

As shown in FIG. 16, the image acquisition apparatus 101 includes a placement unit 121 on which an array plate 107 is placed, an irradiation unit 122 that irradiates the array plate with primary light in order to image a first fluorescence image and a second fluorescence image, a light source 110 that is optically coupled to the irradiation unit and is configured to switch between emitting two primary lights of mutually different wavelengths, a light-collection unit 123 that receives and detects secondary light including fluorescence, an optical system 130 consisting of the irradiation unit 122 and the light-collection unit 123, a placement unit 121, a scanning unit 112-2 that scans the placement unit 121, an apparatus control unit 102 that controls scanning by the scanning unit and irradiation timing of the optical system, and an information acquisition apparatus 11000 for executing a program for executing the method of the present embodiment. The pixel pitch of the acquired fluorescence image of the array plate 107 is 3 μm to 30 μm.

The light-collection unit 123 may be configured to include a photodetector 111 such as a digital camera, a fluorescence microscope, a photomultiplier tube (PMT), or an optical sensor.

The apparatus control unit 102 is constituted by an FPGA, a CPU, a memory, embedded software, and the like. In an internal memory of the apparatus control unit 102, the first fluorescence image 103 and the second fluorescence image 104 acquired by the image acquisition apparatus 101 are stored in file formats such as binary data and 16-bit grayscale Tiff format.

The information acquisition apparatus 11000 is constituted by a general-purpose personal computer or the like, reads the first fluorescence image 103 and the second fluorescence image 104 from the apparatus control unit 102, and executes a program for executing the information acquisition method of the present embodiment. The first fluorescence image 103 and the second fluorescence image 104 and analysis results thereof may be displayed on a user interface 106.

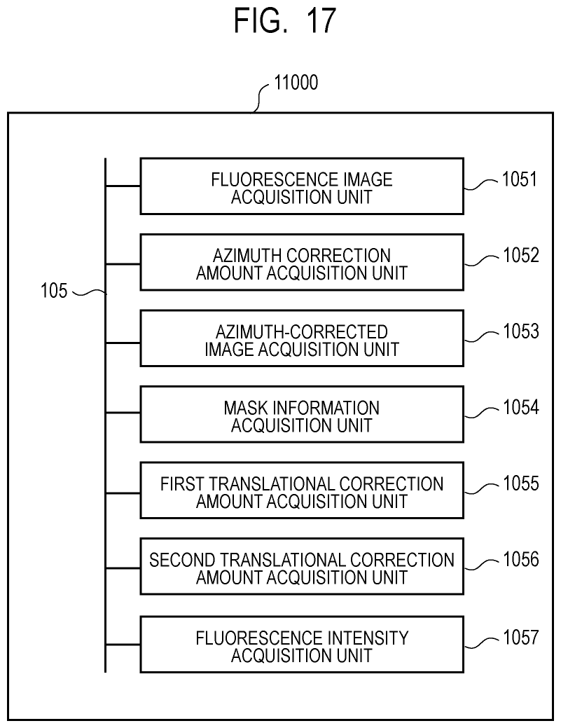

FIG. 17 is a diagram showing a schematic configuration of an information acquisition apparatus 11000 for use in the information acquisition method according to the first embodiment.

As shown in FIG. 17, the information acquisition apparatus 11000 includes a fluorescence image acquisition unit 1051, an azimuth correction amount acquisition unit 1052, an azimuth-corrected image acquisition unit 1053, a template mask information acquisition unit 1054, a first translational correction amount acquisition unit 1055, a second translational correction amount acquisition unit 1056, and a fluorescence intensity acquisition unit 1057.

The fluorescence image acquisition unit 1051, the azimuth correction amount acquisition unit 1052, the azimuth-corrected image acquisition unit 1053, the template mask information acquisition unit 1054, the first translational correction amount acquisition unit 1055, the second translational correction amount acquisition unit 1056, and the fluorescence intensity acquisition unit 1057 operate on an unillustrated processing unit provided in the computer 105. Each of elements 1051 to 1057 described in FIG. 17 constituting the information acquisition apparatus 11000 functionally operates on an unillustrated processing unit such as a CPU, GPU, or ASIC provided in the computer 105. That is, it may also be said that they are executed as software.

The fluorescence image acquisition unit 1051, the azimuth correction amount acquisition unit 1052, the azimuth-corrected image acquisition unit 1053, the template mask information acquisition unit 1054, the first translational correction amount acquisition unit 1055, the second translational correction amount acquisition unit 1056, and the fluorescence intensity acquisition unit 1057 are implemented so that they can provide and read information to and from one another via a signal line 1050. The signal line 1050 can be replaced not only with a signal line on an arithmetic processor but also with a system bus on a motherboard, an Internet line, and the like.

The fluorescence image acquisition unit 1051 acquires, with respect to an array plate provided with a plurality of arranged spots, a first fluorescence image imaged by irradiating first primary light, and a second fluorescence image imaged by irradiating second primary light having a wavelength different from that of the first primary light in order to acquire functional information of the spots.

The azimuth correction amount acquisition unit 1052 acquires information regarding an azimuth correction amount within the image plane of the first fluorescence image and the second fluorescence image based on the first fluorescence image.

The azimuth-corrected image acquisition unit 1053 acquires a first azimuth-corrected image obtained by correcting an azimuth with respect to the first fluorescence image and a second azimuth-corrected image obtained by correcting an azimuth with respect to the second fluorescence image based on the information regarding the azimuth correction amount.

The template mask information acquisition unit 1054 acquires information regarding a template mask including a plurality of first regions arranged corresponding to the plurality of spots, wherein a reference position and relative positions defining an outer edge with respect to the reference position are defined so that one of the plurality of spots overlaps a corresponding first region and another adjacent spot does not overlap the first region.

The first translational correction amount acquisition unit 1055 acquires information regarding a first translational correction amount for the plurality of first regions included in the first azimuth-corrected image by adjusting a relative position of the template mask with respect to the first azimuth-corrected image.

The second translational correction amount acquisition unit 1056 acquires a second translational correction amount for the plurality of first regions included in the second azimuth-corrected image by adjusting a relative position of the template mask with respect to the second azimuth-corrected image based on the information regarding the first translational correction amount for the plurality of first regions included in the first azimuth-corrected image.

The fluorescence intensity acquisition unit 1057 acquires information regarding fluorescence intensity corresponding to the plurality of spots in the first fluorescence image based on the information regarding the first translational correction amount and the first azimuth-corrected image, and acquires information regarding fluorescence intensity corresponding to the plurality of spots in the second fluorescence image based on the information regarding the second translational correction amount and the second azimuth-corrected image.

According to the present embodiment, it becomes possible to configure an information acquisition apparatus that operates fluorescence detection through image analysis in a series and outputs analyzed data.

In the present embodiment, a step of analyzing another fluorescence image relating to functional information of the array plate corresponding to the second fluorescence image may also be further included.

The present disclosure also provides a computer-readable storage medium in which a program causing a computer to execute each of the steps described above is recorded.

### Details of First Embodiment

The information acquisition method S10000 according to the first embodiment will be described in more detail with reference to FIG. 1. The first fluorescence image and the second fluorescence image have been imaged in advance by an image acquisition apparatus such as a fluorescence plate reader.

### Fluorescence Image Acquisition Step S100

This step acquires, with respect to an array plate provided with a plurality of arranged spots, a first fluorescence image imaged by irradiating first primary light, and a second fluorescence image imaged by irradiating second primary light having a wavelength different from that of the first primary light in order to acquire functional information of the spots. The first fluorescence image and the second fluorescence image can be acquired using an image analysis PC.

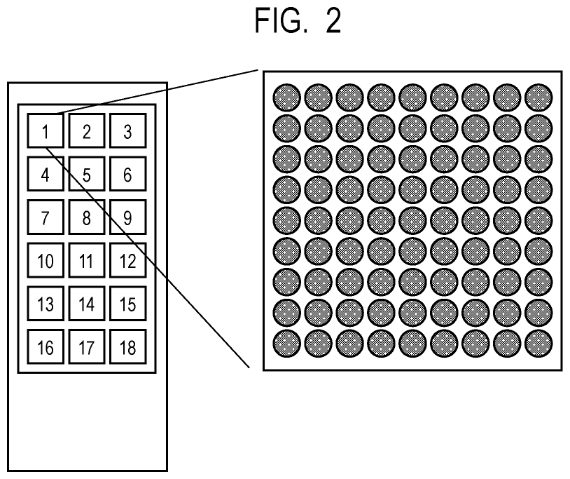

A plurality of kinds of proteins are spotted in an array on the array plate. A schematic diagram of the array plate is shown in FIG. 2. One spot has a diameter of about 100 μm, and the interval between adjacent spots is about 40 μm. The spots are configured in units of 9×9 blocks, and the blocks are arranged in a 3×6 matrix. Spots on the array plate of the present embodiment are fluorescently labeled with Alexa Flour 680. Some protein spots are phosphorylated, and only the phosphorylated protein spots are fluorescently labeled with Alexa Flour 790.

A fluorescence image acquired by exciting this array plate with light having a wavelength of 670 nm is the first fluorescence image.

A fluorescence image acquired by exciting this array plate with light having a wavelength of 780 nm is the second fluorescence image.

It is desirable that the image acquisition apparatus include excitation light of a first wavelength of 670 nm for exciting protein spots fluorescently labeled with Alexa Flour 680. It is also desirable that the image acquisition apparatus include excitation light of a second wavelength of 780 nm for exciting protein spots fluorescently labeled with Alexa Flour 790.

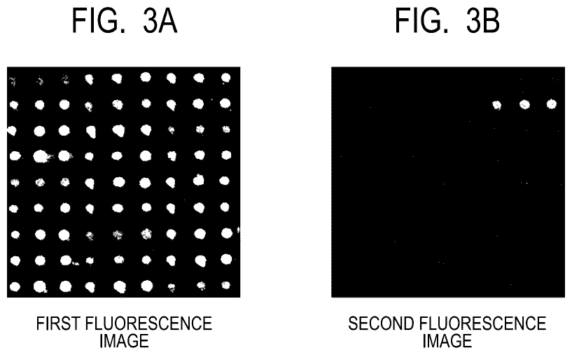

Fluorescence images of the array plate acquired in step S100 are shown in FIGS. 3A and 3B. Here, one block is displayed in an enlarged manner. In the first fluorescence image, all spots are measured uniformly brightly in order to acquire morphological information by irradiating first primary light. In the second fluorescence image, only phosphorylated protein spots are measured brightly in order to acquire functional information by irradiating second primary light.

### Azimuth Correction Amount Acquisition Step S200

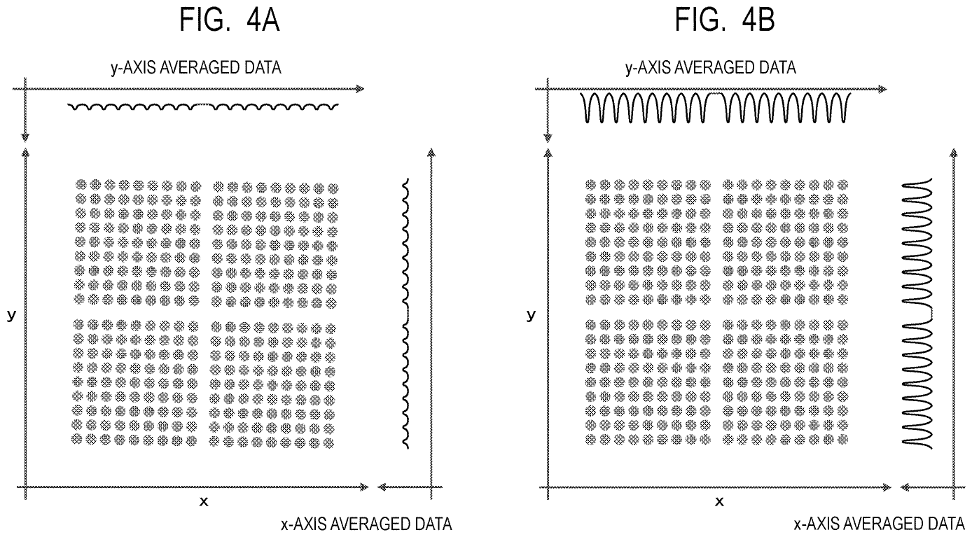

This step acquires information regarding an azimuth correction amount within the image plane of the first fluorescence image and the second fluorescence image obtained in step S100. A method for implementing step S200 will be described with reference to FIGS. 4A and 4B.

By averaging the first fluorescence image obtained by step S100 in an x-axis direction, which is the horizontal direction of the image, and a y-axis direction, which is the vertical direction, one-dimensional x-axis averaged data and y-axis averaged data are calculated. The inclination of the arrangement direction of the array plate with respect to the x-axis and the y-axis defined from the image is considered. When the arrangement direction of the array plate has a predetermined inclination with respect to the x-axis and the y-axis defined from the image, the peak values of intensities of the x-axis averaged data and the y-axis averaged data are lower than when the arrangement direction of the array plate coincides with the x-axis and the y-axis (FIG. 4A). On the other hand, when the arrangement direction of the array plate coincides with the x-axis and the y-axis, the peak values of intensities of the x-axis averaged data and the y-axis averaged data take maximum values (FIG. 4B). From this viewpoint, a temporary azimuth correction amount is given to the fluorescence image to create an azimuth-corrected image, and an azimuth correction amount that maximizes amplitudes of the x-axis averaged data and the y-axis averaged data for the created azimuth-corrected image is acquired.

For example, an angular deviation within the image plane of the first fluorescence image or the second fluorescence image depends on scanning accuracy of the image acquisition apparatus and production accuracy of the array plate, but is about ±1 degree. In the present embodiment, azimuth-corrected images are generated while changing the temporary azimuth correction amount by 0.1 degree at a time, and an azimuth correction amount that maximizes amplitudes of the x-axis averaged data and the y-axis averaged data is acquired.

### Azimuth-Corrected Image Acquisition Step S300

By this step, a first azimuth-corrected image and a second azimuth-corrected image are acquired, in which the first fluorescence image and the second fluorescence image have been corrected with the azimuth correction amount obtained in the azimuth correction amount acquisition step S200. For azimuth correction of the fluorescence images, an affine transformation is used. The affine transformation is executed by any interpolation method such as nearest-neighbor interpolation, linear interpolation, bilinear interpolation, or bicubic interpolation.

### Template Mask Information Acquisition Step S400

This step acquires information regarding a template mask including a plurality of first regions arranged corresponding respectively to the plurality of spots, wherein a reference position and relative positions defining an outer edge with respect to the reference position are defined so that one of the plurality of spots overlaps a corresponding first region and another adjacent spot does not overlap the first region.

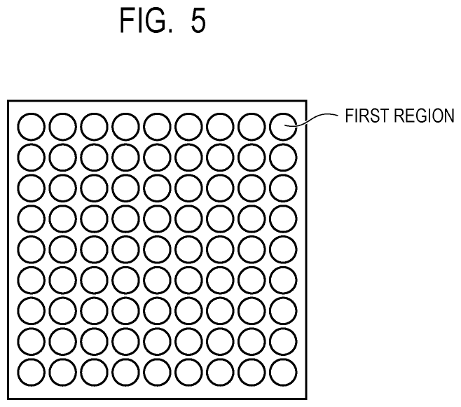

Information regarding the template mask is input in advance based on design information of the array plate by using a prepared user interface. Alternatively, a file created in advance based on design information of the array plate and stored in a memory is read by an analysis PC. The shape of the template mask is shown in FIG. 5. In the template mask, first regions that are regions of interest are arranged in a 9×9 matrix. The shape of a first region is circular with the same radius as that of a spot, in accordance with the shape of a spot on the array plate. Alternatively, the shape of a first region can be any shape within a range that does not overlap another adjacent first region, such as being set slightly larger than the radius of a spot or being made elliptical in accordance with the shape of a spot.

The interval (pitch) between the first regions of the template mask may also be determined from periods of the x-axis averaged data and the y-axis averaged data obtained in step S200.

### First Translational Correction Amount Acquisition Step S500

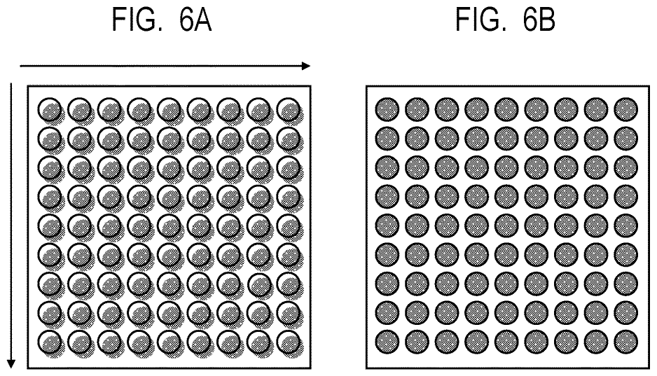

This step acquires information regarding a first translational correction amount for a plurality of first regions included in the first azimuth-corrected image by adjusting a relative position of the template mask with respect to the first azimuth-corrected image. The first translational correction amount acquisition step S500 of the present embodiment will be described with reference to FIGS. 6A and 6B.

Coordinates of each block on the first azimuth-corrected image are set by using x-axis averaged data and y-axis averaged data in the first azimuth-corrected image obtained in step S300.

By changing a relative position of the template mask for a certain block in the x-axis direction and the y-axis direction within a preset scanning range (first scanning range), sums of fluorescence brightness values of the first azimuth-corrected image overlapping a plurality of first regions of the template mask at each relative position are calculated. A relative position at which the sum of the fluorescence brightness values is maximum is acquired as a first translational correction amount in the x-axis direction and the y-axis direction.

As a result, spot positions of the template mask and the block, which had been displaced before scanning as in FIG. 6A, come into alignment by scanning as in FIG. 6B.

For example, when a spot has a diameter of 100 μm, the scanning range of the template mask is preset to 100 μm in each of the x-axis direction and the y-axis direction with respect to the x-axis averaged data and the y-axis averaged data in the first azimuth-corrected image obtained in step S300.

This processing is performed for 18 template masks in total, one for each block.

### Second Translational Correction Amount Acquisition Step S600

This step acquires a second translational correction amount for a plurality of first regions included in the second azimuth-corrected image by adjusting a relative position of the template mask with respect to the second azimuth-corrected image based on information regarding the first translational correction amount.

For a certain block on the second azimuth-corrected image, the relative position of the template mask is changed within a second scanning range that is more limited than the first scanning range, with the first translational correction amount obtained in step S500 being used as a reference position of the relative position of the template mask. At each relative position, sums of fluorescence brightness values of the second azimuth-corrected image overlapping a plurality of first regions of the template mask are calculated. A relative position at which the sum of the fluorescence brightness values is maximum is acquired as a second translational correction amount in the x-axis direction and the y-axis direction.

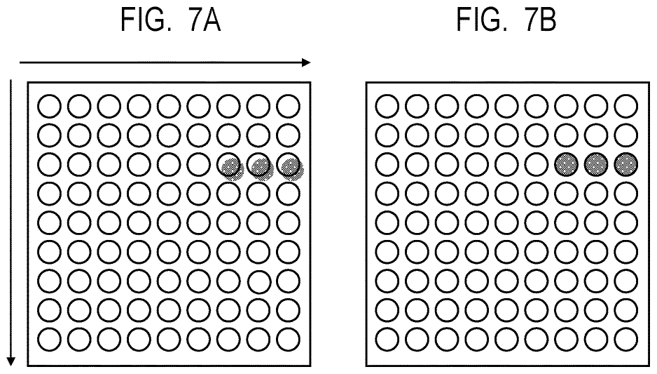

As a result, spot positions of the template mask and the block, which had been displaced before scanning as in FIG. 7A, come into alignment by scanning as in FIG. 7B.

For example, when imaging positions on the array plate at the two wavelengths of the image acquisition apparatus are displaced, a relative positional deviation occurs between the first fluorescence image and the second fluorescence image. According to the present embodiment, even in an image including such a deviation, the positional deviation can be corrected.

If there is no positional deviation or no deviation of the optimum position between the first azimuth-corrected image and the second azimuth-corrected image, the second translational correction amount may have the same value as the first translational correction amount. Further, if the positional deviation or the deviation of the optimum position between the first azimuth-corrected image and the second azimuth-corrected image takes a fixed value, the second translational correction amount may be a value obtained by adding a fixed value to the first translational correction amount.

This processing is performed for 18 template masks in total, one for each block.

### Fluorescence Intensity Acquisition Step S700

This step acquires information regarding fluorescence intensity corresponding to a plurality of spots in the first fluorescence image based on the information regarding the first translational correction amount and the first azimuth-corrected image, and acquires information regarding fluorescence intensity corresponding to a plurality of spots in the second fluorescence image based on the information regarding the second translational correction amount and the second azimuth-corrected image.

Information regarding fluorescence intensity includes information regarding fluorescence intensity (signal) in a plurality of first regions of the template mask, and information regarding fluorescence intensity (background) in a plurality of second regions defined outside the first region so as not to overlap first regions of other adjacent spots.

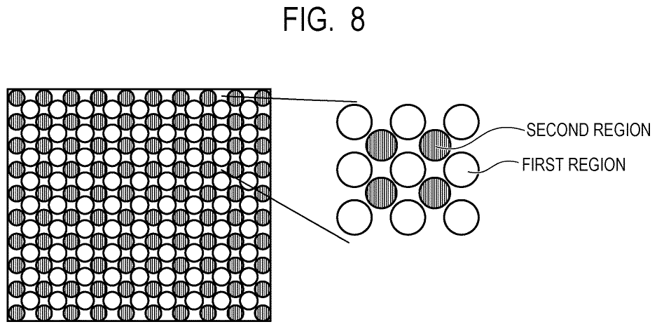

The shape of the second region is shown in FIG. 8 as an example. Second regions corresponding to a first region are defined by four circles that do not overlap a plurality of adjacent first regions and are arranged at equal distances from the first region. The shape of the second region is not limited, and may be a concentric circle having a radius larger than that of the first region, a rectangle, a shape in which adjacent first regions are cut out, or the like.

For each spot, statistical values such as average value, median value, and variance value of the signal and the background, and statistical values of a net signal obtained by extracting a fluorescence brightness component derived from the spot, such as a difference or ratio between the signal and the background, are calculated and output as a file together with data related to the position of the spot.

According to the present embodiment, even when the second fluorescence image is discrete and it is difficult to identify block coordinates, the array plate can be analyzed accurately.

Further, even when there is a relative positional deviation between the first fluorescence image and the second fluorescence image, the position of the template mask is automatically adjusted, and the array plate can be analyzed.

### Second Embodiment

This embodiment is a modified embodiment of the first embodiment, and differs from the first embodiment in that the azimuth-corrected image acquisition step S300 is performed so as to calculate the azimuth correction amount by weighting peak values of intensities of x-axis averaged data and y-axis averaged data.

Due to variations in shapes of spots caused by production accuracy of the array plate, azimuth correction amounts that maximize amplitudes of the x-axis averaged data and the y-axis averaged data may differ. In such a case, in step S200, it is effective to calculate the azimuth correction amount by weighting the peak value of intensity of the x-axis averaged data and the peak value of intensity of the y-axis averaged data.

The shape of a spot tends not to be circular due to the production accuracy of the array plate, and tends to become elliptical with a long axis in the y-axis direction. In this case, since the width of a peak indicating a local maximum of the x-axis averaged data becomes broad, making determination of the peak position difficult, the azimuth correction amount is calculated by giving weight to the y-axis averaged data, which has steep peaks, and calculating a weighted average of the x-axis averaged data and the y-axis averaged data.

According to the present embodiment, it becomes possible to acquire an azimuth correction amount even when there is variation in spot shape due to production accuracy of the array plate.

### Third Embodiment

This embodiment is a modified embodiment of the first embodiment, and differs from the first embodiment in that the second translational correction amount acquisition step S600 is performed so as to determine the second translational correction amount based on the first translational correction amount. Even when two-wavelength fluorescence images of a plate reader have different curvatures, step S600 of the present embodiment is effective.

For example, since Alexa Flour 790 is excited even slightly by an excitation light source of wavelength 670 nm, the second fluorescence image is affected by excitation light of wavelength 670 nm. In order to avoid this effect, the plate reader acquires a fluorescence image by separating excitation wavelengths on outward and return paths when scanning in a short-side direction of the array plate. Depending on scanning accuracy of the scanner, a positional deviation of relative positions of scan lines and curvature of the scan lines in opposite directions may occur between the outward path and the return path, and images may be acquired in which curvature in opposite directions occurs in the fluorescence image at wavelength 670 nm and the fluorescence image at wavelength 780 nm.

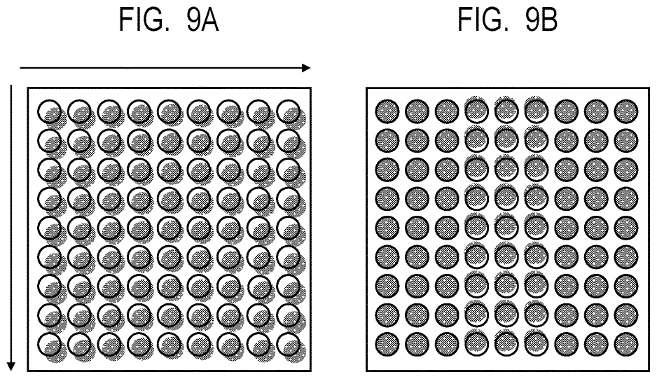

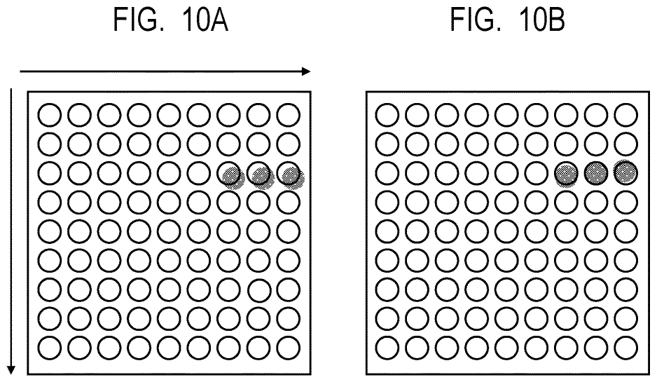

For example, suppose that in step S500 the first fluorescence image is convexly curved as in FIGS. 9A and 9B, and position adjustment of the template mask is performed to acquire the first translational correction amount. In step S600, when the second fluorescence image is concavely curved, the first region of the template mask is displaced with respect to the spot at the same relative position of the template mask. As shown in FIGS. 10A and 10B, by determining the second translational correction amount based on the first translational correction amount, the position of the template mask can be properly adjusted with respect to the second fluorescence image as well.

According to this method, in step S600 the positional deviation of a spot caused by image curvature can be adjusted with the second translational correction amount, and therefore this method is effective even in an image including such curvature.

### Fourth Embodiment

This embodiment is a modified embodiment of the first embodiment, and differs from the first embodiment in that the second translational correction amount acquisition step S600 is performed so as to adjust, with the second translational correction amount, positional deviation of spots derived from the array plate as well.

Step S600 of the present embodiment is effective even when spots occur at different positions in the first fluorescence image that acquires morphological information by irradiating first primary light and the second fluorescence image that acquires functional information by irradiating second primary light due to a production step of the array plate.

For example, suppose that positions at which Alexa Flour 680 and Alexa Flour 790 are fluorescently labeled are different. According to this method, in step S600 the positional deviation of spots derived from the array plate can also be adjusted with the second translational correction amount, and therefore this method is effective even for an array plate including such positional deviation.

### Fifth Embodiment

This embodiment is a modified embodiment of the first embodiment, and differs from the first embodiment in that a noise processing step SS220 is performed after the fluorescence image acquisition step and before the azimuth correction amount acquisition step.

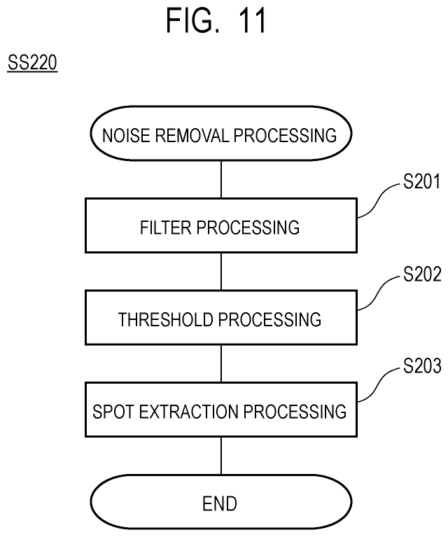

In the first embodiment, it was assumed that the first fluorescence image did not include a noise signal, but when a noise signal is included, it may be difficult to calculate the azimuth correction amount. Unnecessary signals may include a signal-to-background ratio (SN) of a scanner, dust or fluorescent dye that has accidentally adhered to or become mixed in an array plate, and the like. In such a case, the noise removal processing step shown in FIG. 11 is effective. In the present embodiment, that method will be described.

The noise processing step SS220 shown in FIG. 11 can be performed after the fluorescence image acquisition step and before the azimuth correction amount acquisition step. The noise removal processing step includes a filter processing step (S201), a threshold processing step (S202) that reduces the number of gradations related to pixel values, and a spot extraction processing step (S203) that extracts spots. Details of each step will be described below.

### Filter Processing Step S201

With respect to the first fluorescence image obtained by step S100, the effect of system noise imparted to the entire fluorescence image is reduced by performing any filter processing such as a median filter or a Gaussian filter. The filter mask size for filter processing is set to any size so long as the approximate shape of a spot is not affected.

For example, when the scanner has poor SN, high-brightness noise of pixel size may be included even in regions other than spots. Further, low-brightness pixels may occur in a high-brightness region inside a spot. Furthermore, highly bright points having a size of a few pixels, generated because proteins nonspecifically adsorbed outside the spot region are fluorescently labeled, may be included in the fluorescence image. In order to remove these, filter processing is performed with a 5×5-pixel median filter.

### Threshold Processing Step S202

With respect to the first fluorescence image after filter processing obtained by step S201, threshold processing is performed to reduce the number of gradations. For example, in order to distinguish a spot region from a non-spot region, a threshold is determined and the first fluorescence image is converted into a binarized image. The threshold may be input in advance, or may be determined from an algorithm such as Otsu binarization.

### Spot Extraction Processing Step S203

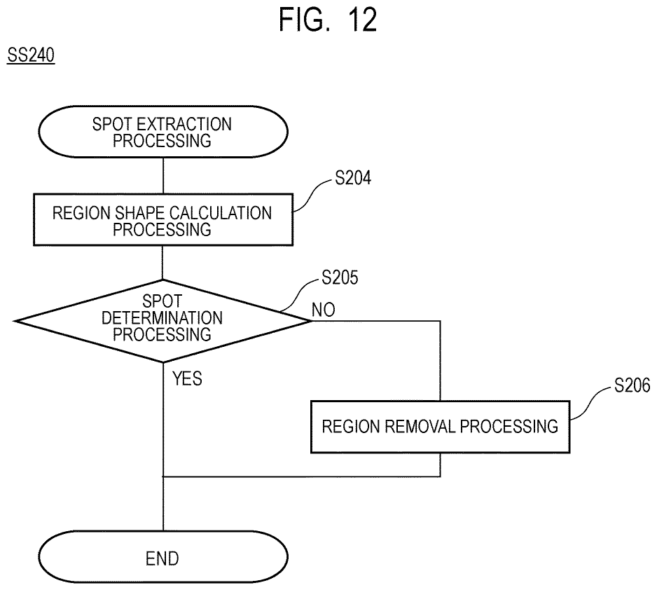

With respect to the first fluorescence image after threshold processing obtained by step S202, spot extraction processing is performed to remove unnecessary signals. The spot extraction processing step SS240 includes a region shape calculation step (S204), a spot determination processing step (S205), and a region removal processing step (S206) shown in FIG. 12. Details of each step will be described below.

### Region Shape Calculation Step S204

With respect to the first fluorescence image after threshold processing obtained by step S202, information regarding shapes of high-brightness regions is acquired, the high-brightness regions being regions in which pixels having the same brightness are adjacent. For example, in a binarized image, brightness values are 0 or 1, and regions of adjacent pixels having brightness value 1 are a plurality of high-brightness regions including spots. As information regarding the shapes of the high-brightness regions, lengths of outer peripheries of the regions, areas of the regions, lengths in a major-axis direction and a minor-axis direction of the regions, and the like are acquired.

### Spot Determination Processing Step S205

On the basis of the information regarding shapes of the high-brightness regions obtained by step S204, it is determined whether a high-brightness region is a spot or is derived from another unnecessary signal. For example, when the length of the outer periphery of a region is larger than a circumferential length calculated from a spot diameter in design of the array plate, the region is determined not to be a spot. Further, whether a high-brightness region is a spot is determined by determination expressions such as an expression in which circularity calculated from the length of the outer periphery of the region and the area is greater than a preset circularity, and an expression in which a ratio between the length in the major-axis direction and the length in the minor-axis direction is greater than a preset value.

### Region Removal Processing Step S206

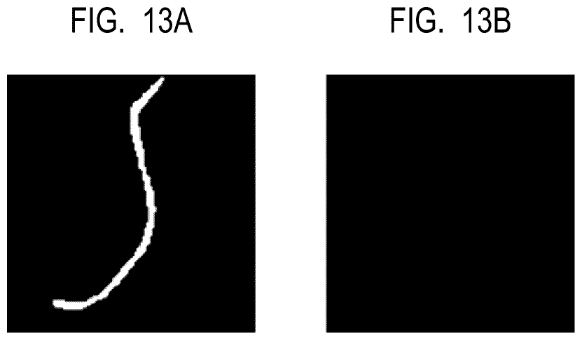

A pixel value of a low-brightness region is assigned to a high-brightness region that has been determined not to be a spot by step S205. For example, when fibrous dust as shown in FIG. 13A is included in the binarized first fluorescence image as an unnecessary high-brightness signal, as shown in FIG. 13B, the brightness value of the high-brightness region is converted from 1 to 0.

The noise removal processing step according to the present embodiment is executed after S100 of the first embodiment and before S200. A noise removal image is acquired by removing noise from the first fluorescence image by the noise removal processing step. Subsequent steps are the same as in the first embodiment. In step S200, information regarding the azimuth correction amount is acquired by using the noise removal image instead of the first fluorescence image. According to the present embodiment, it becomes possible to calculate the azimuth correction amount without depending on the SN of the scanner or the state of the array plate.

### Sixth Embodiment

This embodiment is a modified embodiment of the first embodiment, and differs from the first embodiment in that, in the first embodiment, information regarding the azimuth correction amount within the image plane of the first fluorescence image and the second fluorescence image is acquired based on the first fluorescence image, whereas in the present embodiment, a composite image acquisition step of acquiring a composite image of the first fluorescence image and the second fluorescence image is performed in order to calculate the azimuth correction amount.

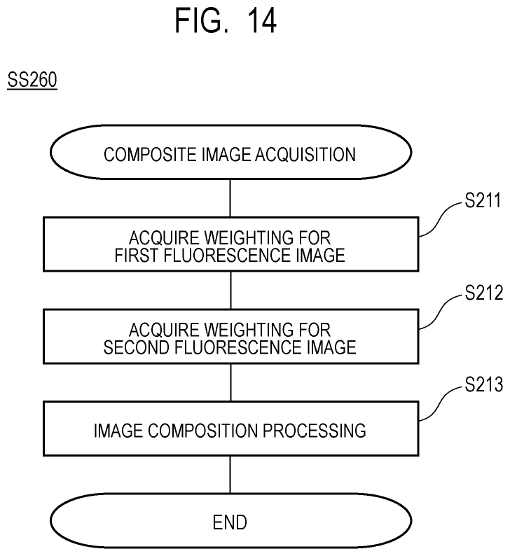

The composite image acquisition step SS260 according to the sixth embodiment will be described with reference to FIG. 14.

In the first embodiment, the azimuth correction amount is calculated from the first fluorescence image, but depending on measurement parameters at the time of imaging the fluorescence image, contrast between spots and areas outside the spots in the first fluorescence image may be low, making it difficult to calculate the azimuth correction amount. In such a case, it is effective to calculate the azimuth correction amount from a composite image of the first fluorescence image and the second fluorescence image obtained in the composite image acquisition step. The composite image acquisition step can be performed after the fluorescence image acquisition step and before the azimuth correction amount acquisition step. The process of the composite image acquisition step SS260 is shown in FIG. 14.

The composite image acquisition step includes a step (S211) of acquiring weighting for the first fluorescence image, a step (S212) of acquiring weighting for the second fluorescence image, and an image composition processing step (S213).

By reading preset weighting values in step S211 and step S212, a composition ratio at the time of image composition is acquired. For example, a threshold is determined by Otsu binarization processing, and a pixel value on a high-brightness side is assigned as 1 and a pixel value on a low-brightness side is assigned as 0. At this time, in a fluorescence image having high contrast, a spot region has a pixel value of 1 and an area outside the spot region has a pixel value of 0. On the other hand, in a fluorescence image having low contrast, the pixel value becomes 1 regardless of whether it is inside or outside a spot region, and variation in an in-plane distribution of pixels having pixel value 1 becomes large. By using this characteristic of the binarized image, it is effective to increase a weight when variation in the in-plane distribution of pixels having pixel value 1 is small.

In step S213, on the basis of the obtained composition ratio, a composition method such as additive averaging composition or lighten composition is set so that contrast between spots and areas outside the spots becomes high.

The noise removal processing step of FIG. 11 may be applied to a composite image or a fluorescence image before or after the composite image acquisition step.

According to the present embodiment, the azimuth correction amount can be calculated even when contrast between spots and areas outside the spots in the first fluorescence image is low.

### Seventh Embodiment

This embodiment is a modified embodiment of the first embodiment, and differs from the first embodiment in that, in the first embodiment, the azimuth correction amount was calculated from peak values of intensities of x-axis averaged data and y-axis averaged data, whereas in the present embodiment, the azimuth correction amount is calculated from positions of local peaks appearing in a matrix in a two-dimensional frequency image. A method different from the first embodiment for implementing step S200 of FIG. 1 in the seventh embodiment will be described.

In the first embodiment, the azimuth correction amount was calculated from peak values of intensities of x-axis averaged data and y-axis averaged data, but in the azimuth correction amount acquisition step, information regarding the azimuth correction amount can also be acquired by performing two-dimensional frequency analysis of the first fluorescence image.

In the present embodiment, the azimuth correction amount is calculated from a two-dimensional frequency image obtained by a two-dimensional Fourier transform of the first fluorescence image. The two-dimensional frequency image is an image in which complex amplitudes in a frequency space after the two-dimensional Fourier transform are expressed by absolute values. Specifically, the azimuth correction amount is calculated from positions of local peaks appearing in a matrix in the two-dimensional frequency image.

The present embodiment has an advantage in that, whereas in the first embodiment it was necessary to create azimuth-corrected images by temporary azimuth correction amounts a plurality of times, in the present embodiment it is only necessary to generate the two-dimensional frequency image once.

### Eighth Embodiment

This embodiment is a modified embodiment of the first embodiment, and differs from the first embodiment in that, in the first embodiment, a relative position of the template mask at which fluorescence brightness values of the azimuth-corrected image in a region overlapping a plurality of first regions of the template mask are maximum was acquired as a translational correction amount, whereas in the present embodiment, a relative position of the template mask at which the sum of fluorescence brightness values of the azimuth-corrected image in regions overlapping the second regions in step S700 of the first embodiment is minimized is calculated as a translational correction amount.

In the eighth embodiment, a method for implementing steps corresponding to S500 and S600 of FIG. 1 will be described.

In the first embodiment, a relative position of the template mask at which fluorescence brightness values of the azimuth-corrected image in a region overlapping a plurality of first regions of the template mask are maximum was acquired as a translational correction amount.

In contrast, in the present embodiment, a relative position of the template mask at which the sum of fluorescence brightness values of the azimuth-corrected image in regions overlapping the second regions in step S700 of the first embodiment is minimized is calculated as a translational correction amount.

According to the present embodiment, it becomes possible to calculate a translational correction amount even when the first region of the template mask is larger than a spot.

### Ninth Embodiment

This embodiment is a modified embodiment of the first embodiment, and differs from the first embodiment in that, in the first embodiment, a relative position of the template mask at which fluorescence brightness values of the azimuth-corrected image in a region overlapping a plurality of first regions of the template mask are maximum was acquired as a translational correction amount, whereas in the present embodiment, a mask image is created from arrangement information of the template mask, and a relative position at which two-dimensional cross-correlation between the azimuth-corrected image and the mask image shifted in relative position becomes maximum is calculated as a translational correction amount.

In the ninth embodiment, a method for implementing steps S500 and S600 of FIG. 1 will be described.

In the first embodiment, a relative position of the template mask at which fluorescence brightness values of the azimuth-corrected image in a region overlapping a plurality of first regions of the template mask are maximum was acquired as a translational correction amount. Depending on the fluorescence image, a highly bright signal other than a spot may be included in the vicinity of a spot, and a maximum value search is affected thereby.

In the present embodiment, a mask image is created from arrangement information of the template mask, and a relative position at which two-dimensional cross-correlation between the azimuth-corrected image and the mask image shifted in relative position becomes maximum is calculated as a translational correction amount.

According to the present embodiment, it becomes possible to calculate a translational correction amount while reducing an effect of a highly bright unnecessary signal included in the fluorescence image.

### Tenth Embodiment

This embodiment is a modified embodiment of the first embodiment, and differs from the first embodiment in that, in the first embodiment, a translational correction amount was calculated for each template mask, whereas in the present embodiment, after the translational correction amount is calculated, a fine translational correction amount for fine adjustment is calculated for each spot.

In the tenth embodiment, a method for implementing steps S500 and S600 of FIG. 1 will be described.

In the first embodiment, a translational correction amount was calculated for each template mask, but due to e.g. variation in spot shape caused by production accuracy of the array plate and image curvature caused by scanning accuracy of the scanner, spots may occur that do not fit correctly within the first region of the template mask.

In the present embodiment, after the translational correction amount is calculated in step S500 and step S600, a fine translational correction amount for fine adjustment is calculated for each spot. A fine translational correction amount is acquired by scanning the first region so that a brightness value inside the first region of the template mask for each spot becomes maximum.

According to the present embodiment, it becomes possible to set the first region of the template mask correctly even when there is variation in spot shape or image curvature caused by scanning accuracy of the scanner.

### Eleventh Embodiment

This embodiment is a modified embodiment of the first to tenth embodiments, and differs from the first to tenth embodiments in that, in the first to tenth embodiments, there was one fluorescence image imaged by irradiating second primary light for acquiring functional information of a spot, whereas in the present embodiment a plurality of fluorescence images corresponding to the second fluorescence image are acquired.

In the first to tenth embodiments, there was one fluorescence image imaged by irradiating second primary light for acquiring functional information of a spot.

In the present embodiment, a plurality of fluorescence images corresponding to the second fluorescence image, which is a fluorescence image for acquiring functional information of a spot and is imaged by irradiating second primary light, are acquired.

According to the present embodiment, functional information other than phosphorylation information can be acquired simultaneously.

According to the above embodiments of the present disclosure, it is possible to provide an information acquisition method and an information acquisition apparatus that acquire information regarding fluorescence intensity with ensured reproducibility in each of a fluorescence image having morphological information of spots on an array plate and a fluorescence image having functional information.

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. An information acquisition method for acquiring information regarding fluorescence intensity of spots arranged on an array plate, the method comprising:
a fluorescence image acquisition step of acquiring, with respect to the array plate provided with a plurality of arranged spots, a first fluorescence image imaged by irradiating first primary light, and a second fluorescence image imaged by irradiating second primary light having a wavelength different from that of the first primary light in order to acquire functional information of the spots;
an azimuth correction amount acquisition step of acquiring information regarding an azimuth correction amount within the image plane of the first fluorescence image and the second fluorescence image based on the first fluorescence image;
an azimuth-corrected image acquisition step of acquiring a first azimuth-corrected image obtained by correcting an azimuth with respect to the first fluorescence image and a second azimuth-corrected image obtained by correcting an azimuth with respect to the second fluorescence image based on the information regarding the azimuth correction amount;
a template mask information acquisition step of acquiring information regarding a template mask including a plurality of first regions arranged corresponding respectively to the plurality of spots, wherein a reference position and relative positions defining an outer edge with respect to the reference position are defined so that one of the plurality of spots overlaps a corresponding first region and another adjacent spot does not overlap the first region;
a first translational correction amount acquisition step of acquiring information regarding a first translational correction amount for the plurality of first regions included in the first azimuth-corrected image by adjusting a relative position of the template mask with respect to the first azimuth-corrected image;
a second translational correction amount acquisition step of acquiring a second translational correction amount for the plurality of first regions included in the second azimuth-corrected image by adjusting a relative position of the template mask with respect to the second azimuth-corrected image based on the information regarding the first translational correction amount for the plurality of first regions included in the first azimuth-corrected image; and
a fluorescence intensity acquisition step of acquiring information regarding fluorescence intensity corresponding to the plurality of spots in the first fluorescence image based on the information regarding the first translational correction amount and the first azimuth-corrected image, and acquiring information regarding fluorescence intensity corresponding to the plurality of spots in the second fluorescence image based on the information regarding the second translational correction amount and the second azimuth-corrected image.

2. The information acquisition method according to claim 1, further comprising, after the fluorescence image acquisition step and before the azimuth correction amount acquisition step, a noise removal processing step of performing filter processing on the first fluorescence image, threshold processing for reducing the number of gradations related to pixel values, spot extraction processing for extracting spots, or acquiring a noise removal image.

3. The information acquisition method according to claim 1, further comprising, after the fluorescence image acquisition step and before the azimuth correction amount acquisition step, a composite image acquisition step of acquiring a composite image of the first fluorescence image and the second fluorescence image.

4. The information acquisition method according to claim 1, wherein in the azimuth correction amount acquisition step, information regarding the azimuth correction amount is acquired by performing two-dimensional frequency analysis of the first fluorescence image.

5. The information acquisition method according to claim 1, wherein the first translational correction amount acquisition step includes a step of acquiring information regarding the first translational correction amount based on fluorescence intensity in a plurality of second regions defined outside the first regions of the template mask so as not to overlap first regions of other adjacent spots.

6. The information acquisition method according to claim 1, wherein, in place of the first translational correction amount acquisition step and the second translational correction amount acquisition step, the method includes a step of adjusting relative positions of the template mask relating to the first translational correction amount and the second translational correction amount, adjusting relative positions of the plurality of first regions included in the first azimuth-corrected image to acquire a plurality of first fine translational correction amounts for the first regions, and adjusting relative positions of the plurality of first regions included in the second azimuth-corrected image to acquire information regarding a plurality of second fine translational correction amounts for the first regions.

7. The information acquisition method according to claim 1, wherein the first fluorescence image includes morphological information of the spots, and the second fluorescence image includes functional information of the spots.

8. Computer-readable non-transitory storage medium in which a program causing a computer to execute each step of the information acquisition method according to claim 1 is recorded.

9. An image acquisition apparatus comprising: a computer comprising a processor and a memory storing instructions that cause the processor to execute each step of the information acquisition method according to claim 1; an optical system including an irradiation unit that irradiates the array plate with primary light and a light-collection unit that collects secondary light from the spots; a placement unit on which the array plate is placed; a scanning unit that relatively moves the placement unit and the optical system;
and a control unit that controls scanning by the scanning unit and irradiation timing of the optical system.

10. The image acquisition apparatus according to claim 9, further comprising a light source optically coupled to the irradiation unit.

11. The image acquisition apparatus according to claim 10, wherein the light source is configured to switch between emitting the first primary light and the second primary light.

12. An information acquisition apparatus for acquiring information regarding fluorescence intensity of spots arranged on an array plate, the apparatus comprising:
a fluorescence image acquisition unit that acquires, with respect to the array plate provided with a plurality of arranged spots, a first fluorescence image imaged by irradiating first primary light, and a second fluorescence image imaged by irradiating second primary light having a wavelength different from that of the first primary light in order to acquire functional information of the spots;
an azimuth correction amount acquisition unit that acquires information regarding an azimuth correction amount within the image plane of the first fluorescence image and the second fluorescence image based on the first fluorescence image;
an azimuth-corrected image acquisition unit that acquires a first azimuth-corrected image obtained by correcting an azimuth with respect to the first fluorescence image and a second azimuth-corrected image obtained by correcting an azimuth with respect to the second fluorescence image based on the information regarding the azimuth correction amount;
a template mask information acquisition unit that acquires information regarding a template mask including a plurality of first regions arranged corresponding respectively to the plurality of spots, wherein a reference position and relative positions defining an outer edge with respect to the reference position are defined so that one of the plurality of spots overlaps a corresponding first region and another adjacent spot does not overlap the first region;
a first translational correction amount acquisition unit that acquires information regarding a first translational correction amount for the plurality of first regions included in the first azimuth-corrected image by adjusting a relative position of the template mask with respect to the first azimuth-corrected image;
a second translational correction amount acquisition unit that acquires a second translational correction amount for the plurality of first regions included in the second azimuth-corrected image by adjusting a relative position of the template mask with respect to the second azimuth-corrected image based on the information regarding the first translational correction amount for the plurality of first regions included in the first azimuth-corrected image; and
a fluorescence intensity acquisition unit that acquires information regarding fluorescence intensity corresponding to the plurality of spots in the first fluorescence image based on the information regarding the first translational correction amount and the first azimuth-corrected image, and acquires information regarding fluorescence intensity corresponding to the plurality of spots in the second fluorescence image based on the information regarding the second translational correction amount and the second azimuth-corrected image.
